(19) United States
(12) Patent Application Publication
(10) Pub. No.: US 2008/0103485 A1
(43) Pub. Date: May 1, 2008

(54) NON-DISCONNECTABLE POSITIVE LUER-LOCK CONNECTOR

(76) Inventor: Peter Kruger, Damlos (DE)

Correspondence Address: Richard S. Roberts;Roberts & Roberts, L.L.P., Attorneys at Law, P.O. Box 484, Princeton, NJ, 08542-0484, US

(21) Appl. No.: 11/924,818

(22) Filed: Oct. 26, 2007

(30) Foreign Application Priority Data
Oct. 31, 2006 (EP) 06 022 700.2

(51) Int. Cl. A61M 39/10

(52) U.S. Cl. 604/533

(57) ABSTRACT

The invention concerns a positive Luer-lock connector to connect all standardised negative Luer-lock connectors with a Luer taper, a rear shaft joining the Luer taper and a lock's thread. Such detachable connectors are available with various designs. Especially when administering toxic and generally environmentally hazardous media, like for example cytostatic agents, it is desirable to prevent any disconnection of the Luer-lock connection, so that neither patients, nor personnel would get into contact with it. According to the invention this is achieved by that the connector has cogs effectively joined with the lock's thread and that a capping sleeve is placed undetachably from and rotatably on the shaft, the capping sleeve having on its inside counter-cogs matching the cogs, while the cogs and counter-cogs are so constructed, that in a first direction of rotation they engage one another in an arresting manner so that to lock the Luer-lock connection of the capping sleeve on the shaft, in a second direction of rotation, that is opposing the first direction of rotation, they slide off one another in a free-wheeling manner.

The invention concerns a positive Luer-lock connector as well as a needle holder or catheter that has the positive Luer-lock connector.

Such positive (male) Luer-lock connectors or adapters according to DIN EN 1707 are known with the most varied embodiments. They are usually detachable. Especially when administering toxic and generally environmentally hazardous media, like for example cytostatic agents, it is desirable to prevent any disconnection of the Luer-lock connection, so that neither patients, nor personnel would get into contact with the hazardous medium, what could happen due to disconnected connections. In this respect there is the need for a positive Luer-lock adapter or connector, that effectively prevents an intentional or unintentional disconnection of the joint and excludes any dangerous situation.

According to the invention this objective is achieved or the problem is eliminated by that the connector has cogs effectively joined with the lock's thread and that a capping sleeve is placed undetachably from and rotatably on the shaft, the capping sleeve having on its inside counter-cogs matching the cogs, while the cogs and counter-cogs are so constructed, that in a first direction of rotation they engage one another in an arresting manner so that to lock the Luer-lock connection of the capping sleeve on the shaft, in a second direction of rotation, that is opposing the first direction of rotation, they slide off one another in a free-wheeling manner.

According to the invention the special geometric contour of the connector ensures that a reliable liquid-tight and/or gas-tight joint remains permanently and a disconnection can be precluded. The cogs provided in accordance with the invention on the lock's thread and the counter-cogs on the inside of the capping sleeve make a tightening of the connector, for example, by turning it to the right, possible while an unscrewing in the opposite direction of rotation is prevented by the free-wheeling of the cogs and counter-cogs.

The invention also concerns a needle holder or catheter, that has the positive Luer-lock connector constructed according to the invention.

Further advantages and features become obvious from the sub-claims 2 to 7, which may also have inventive significance in conjunction with the main claim.

In the following two preferred embodiments are explained in detail based on the figures, to which, however, the invention is not limited. They merely serve the purpose of better understanding of the invention and its operation. They show in:

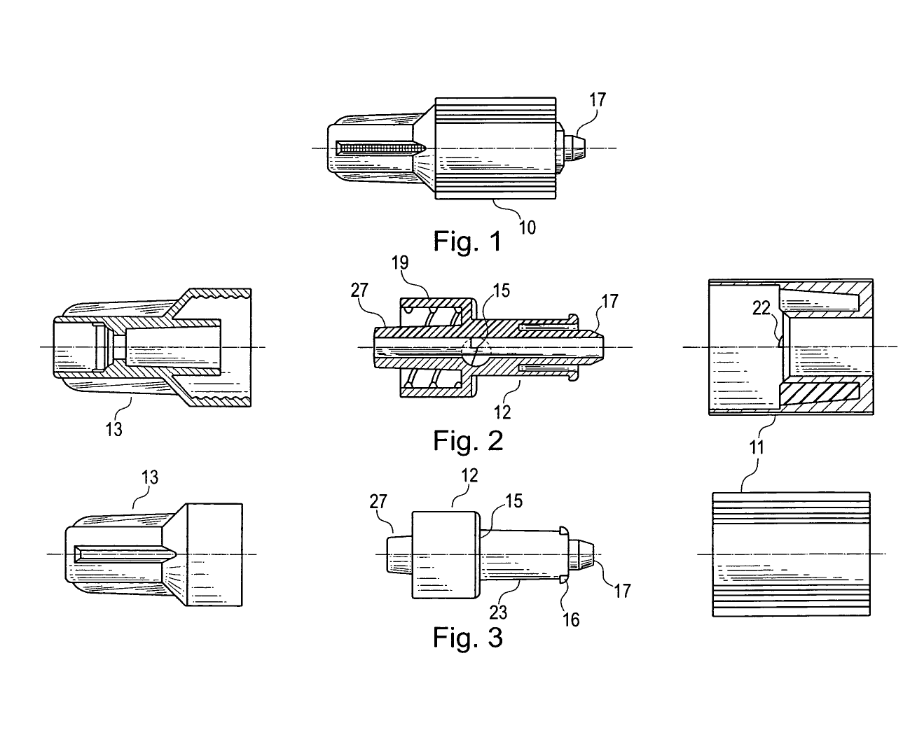

FIG. 1—a side view of an assembled needle holder according to the invention with a protective cap;

FIG. 2—a schematic, three-piece sectioned view in the form of an exploded illustration of the single components of the needle holder according to the invention as shown in FIG. 1;

FIG. 3—side views of the sectioned views of the single components shown in FIG. 2;

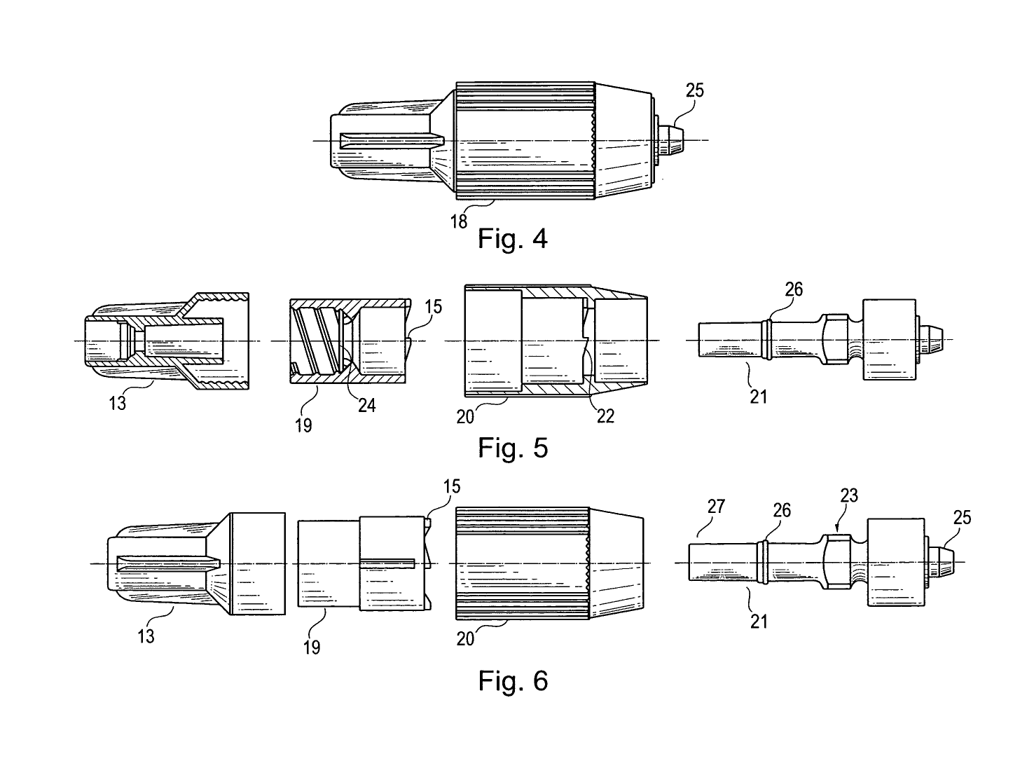

FIG. 4—a side view of a further embodiment of an assembled needle holder according to the invention with a protective cap;

FIG. 5—schematic sectioned side views in the form of an exploded illustration of the needle holder shown in FIG. 4 with its single components; and

FIG. 6—side views of the single components of the needle holder shown in FIG. 5.

In FIGS. 1 and 4 an embodiment each of a needle holder according to the invention is shown, that is designated by 10 and 18, respectively, and has its final non-disconnectable form. Whereas the first embodiment with the protective cap 13 is a three-piece construction, the second embodiment of the needle holder according to the invention with the protective cap 13 is made up from four parts. As far as the invention is concerned the protective cap 13 is of no particular significance. At the right end is the connector 17 and 25, respectively, for a hose (not illustrated). The left end of the needle holder 10 and 18, respectively, is formed by the protective cap 13, that functions as a flow stopper. The knurled capping sleeve 11 and 20, respectively, is provided approximately in the middle. The relative sizes of the single components, which will be explained in detail below, are the usual ones, just as the plastic material, that can be chosen, for example, from ABS, polycarbonate, polyethylene or polyamide. Other materials are conceivable by the person skilled in the art. In the following the particular construction of the positive Luer-lock connector 12 and Luer connector 21, respectively, will be explained, based on FIGS. 2 and 3 and FIGS. 5 and 6, respectively.

Of particular significance for the first embodiment according to the invention, shown in FIGS. 2 and 3, are the cogs 15 connected with the thread 19 in the Luer-lock connector 12 according to the invention, over which a capping sleeve or capping nut 11 is placed, that can rotate around the shaft, but cannot be pulled off it. Counter-cogs 22, matching the cogs 15, are provided inside the externally knurled capping sleeve 11. The cogs 15 and 22 engage one another in the first direction of rotation, whereas in the opposite direction of rotation they slide off one another in a free-wheeling manner. The latter is accomplished due to continuous and edgeless oblique flanks, which merge into a base surface. The capping sleeve 11 no longer can be pulled off the rear shaft 23, because a continuous locking projection 16 is provided in its end region.

By referring to FIGS. 5 and 6 a second embodiment of the invention is explained in detail. Whereas in the first embodiment the lock's thread 19 is firmly joined with the Luer taper 27 and the rear shaft 23 and thus has an integral construction, in the second embodiment the lock's thread 19 is provided in a separate part and is placed freely rotatably on the rear shaft 23 of the positive Luer-lock connector 21. The cogs 15 on the lock's thread 19 interact in this embodiment in the same manner with the counter cogs 22 in the capping nut 20 as was the case in the first embodiment. The arrest of the positive Luer-lock connector 21 is carried out by a snap-in connection, whereby an annular projection 26 on the shaft 23 interacts with a matching recess 24, that is provided continuously approximately in the middle of the lock's thread 19. The snap-in connection ensures that after having been assembled the lock's thread 19 no longer can be pulled off the connector 21. By virtue of this the disconnection of the needle holder 18 will be prevented, since the rotatable lock's thread is joined with the connector 21 in a non-disconnectable manner by means of the snap-in connection. When compared with the first embodiment, an advantage of this is that during the tightening the connector 21 does not rotate, thus preventing the twisting of the transfer hose (not illustrated) when the equipment is being connected in the axial direction that would result its possible bending leading to the endangering of the patient.

1. A positive Luer-lock connector to connect negative Luer-lock connectors having a Luer taper, a rear shaft joining the Luer taper and a lock's thread comprising cogs joined with the lock's thread and a capping sleeve placed undetachably from and rotatably on the shaft, the capping sleeve having on its inside counter-cogs matching the cogs, the cogs and counter-cogs being so constructed, that in a first direction of rotation they engage one another in an arresting manner to lock the Luer-lock connection of the capping sleeve on the shaft, and in a second direction of rotation, that is opposing the first direction of rotation, they slide off one another in a free-wheeling manner.

2. The positive Luer-lock connector according to claim 1, wherein the cogs and the counter-cogs have opposite directed engagement surfaces, which with continuous and edgeless oblique flanks merge rearward into a base surface.

3. The positive Luer-lock connector according to claim 1, wherein the cogs are provided on a surface that is perpendicular to a longitudinal axis of the rear shaft rearward to the cogs and the counter-cogs are provided on an end face situated inside the capping sleeve.

4. The positive Luer-lock connector according to claim 1 wherein the lock's thread is firmly joined with the Luer taper and/or the rear shaft.

5. The positive Luer-lock connector according to claim 1 wherein the lock's thread is formed in a separate capping sleeve that is placed on the rear shaft and can freely rotate on it.

6. The positive Luer-lock connector according to claim 1 wherein on the rear shaft and/or on the capping sleeve locking means are provided for the undetachable locking of the capping sleeve on the rear shaft.

7. The positive Luer-lock connector according to claim 1 which comprises a plastic material.

8. A needle holder comprising the positive Luer-lock connector according to claim 1 at one end and a connector for a transfer hose at its other end.

9. The needle holder according to claim 8, comprising a protective cap placed on the Luer taper of the positive Luer-lock connector.

10. A catheter comprising the positive Luer-lock connector according to claim 1 at one end and a connector for a transfer hose at its other end.

11. The catheter according to claim 10, comprising a protective cap placed on the Luer taper of the positive Luer-lock connector.

12. The positive Luer-lock connector according to claim 1 which comprises ABS or polycarbonate.

13. The positive Luer-lock connector according to claim 1 wherein the lock's thread is integrally constructed with the Luer taper and/or the rear shaft.

14. The positive Luer-lock connector according to claim 2 wherein the lock's thread is firmly joined with the Luer taper and/or the rear shaft.

15. The positive Luer-lock connector according to claim 3 wherein the lock's thread is firmly joined with the Luer taper and/or the rear shaft.

16. The positive Luer-lock connector according to claim 2 wherein the lock's thread is formed in a separate capping sleeve that is placed on the rear shaft and can freely rotate on it.

17. The positive Luer-lock connector according to claim 3 wherein the lock's thread is formed in a separate capping sleeve that is placed on the rear shaft and can freely rotate on it.

18. The positive Luer-lock connector according to claim 2 wherein on the rear shaft and/or on the capping sleeve locking means are provided for the undetachable locking of the capping sleeve on the rear shaft.

19. The positive Luer-lock connector according to claim 3 wherein on the rear shaft and/or on the capping sleeve locking means are provided for the undetachable locking of the capping sleeve on the rear shaft.

20. The positive Luer-lock connector according to claim 4 wherein on the rear shaft and/or on the capping sleeve locking means are provided for the undetachable locking of the capping sleeve on the rear shaft.
